(19) United States
(12) Patent Application Publication
(10) Pub. No.: US 2022/0095995 A1
(43) Pub. Date: Mar. 31, 2022

(54) DEVICE AND METHOD FOR MEASURING CERVICAL DILATION

(71) Applicant: Frotek LLC, Miramar, FL (US)

(72) Inventors: Farhan Naviwala, Miramar, FL (US); Rafael Carreno, Miami, FL (US)

(21) Appl. No.: 17/366,150

(22) Filed: Jul. 2, 2021

Related U.S. Application Data
(60) Provisional application No. 63/047,770, filed on Jul. 2, 2020.

(51) Int. Cl. A61B 5/00; H04N 7/18; A61B 5/107; A61B 1/045; A61B 1/06; H04N 13/239; A61B 1/00; A61B 1/303

(52) U.S. Cl. CPC A61B 5/435; H04N 7/181; A61B 5/1076; A61B 5/1079; A61B 1/303; A61B 1/0676; H04N 13/239; A61B 1/00048; A61B 1/045

(57) ABSTRACT

A optical system for measuring cervical dilation. The device of the subject invention can be a mono-view and/or stereo-view and can include computer enhancement features to provide two-dimensional (2D) and/or three-dimensional (3D) imaging, depth sensing, viewing, and/or measurement capability.

### CROSS-REFERENCE TO RELATED APPLICATIONS

This application is related to, co-pending with, and claims the benefit of the filing date of U.S. Provisional Application Ser. No. 63/047,770 titled “DEVICE AND METHOD FOR MEASURING CERVICAL DILATION” and filed Jul. 2, 2020, and the subject matter of which is incorporated herein by reference.

### STATEMENT REGARDING FEDERALLY SPONSORED RESEARCH OR DEVELOPMENT

Not applicable

### INCORPORATION BY REFERENCE OF MATERIAL SUBMITTED ON A COMPACT DISC

Not applicable

### TECHNICAL FIELD

The present invention relates to the field of cervix measuring, and more specifically, to measuring a cervix for dilation.

### BACKGROUND OF THE INVENTION

Cervical dilation is the most important indicator of labor onset and progression.

The cervix is typically 2 to 3 centimeters in length (about 1 inch) and roughly cylindrical inshape, which changes during pregnancy. The cervix is located at the back of the vagina and is not directly visualized, requiring use of a speculum inserted into the vagina to examine the cervix. During labor, the cervix opens to accommodate the passage of baby's head into the vagina, which is around 10 centimeters (cm) dilated for most term babies.

If the cervix is not dilated, a woman is not in labor. But if her cervix is opening at a steady, regular rate, she is in active labor and getting closer to delivering her baby.

Digital exams are the standard of care to measure cervical dilation. Digital exams are poorly reproducible and time consuming for the clinician; and they are uncomfortable for the patient. According to studies, the overall accuracy for determining the exact diameter of cervical dilation is 56.3%. Intra-observer variability (variation observer experiences when observing the same material more than once) for a given measurement is estimated at about 52%.

Mechanical cervimeters are known. For example, a caliper-type mechanical cervimeter with an integrated ruler has been used since 1956. However, these mechanical cervimeters are inaccurate for dilation measurement greater than 7 cm, lack a recording module, are invasive, and their heavy metallic structure can interfere with the dilation during measurement.

Similar to mechanical cervimeters, electromechanical and electromagnetic cervimeters have also been developed and known since the mid-1950's. These add integrated sensors to convert continuous system movements into electrical signals that can be read and recorded.

Ultrasound cervimeters are another class of cervimeters and have been used since the 1970's. These devices employ ultrasound (US) transducers to measure cervical dilation. Cervical length (CL) by transvaginal ultrasound (TVUS) is routinely used to predict preterm labor, it is also used to predict spontaneous labor in prolonged pregnancies and in the predication of successful labor induction.

Researchers at Duke University have developed a probe insertable within the vagina to view and detect cervical cancer. The device is sterilizable and re-useable, and comprises a light source and a camera having a zoom feature for enlarging the viewing field. This device is not described as being useful for measuring cervical dilation.

While multiple tools have been conceived and developed over the years, nothing has gained traction to replace digital exams.

There is a need for an accurate and reproducible cervical dilation measurements free from human error and variability to determine the onset and progression of labor. There is an unmet need for a reliable way to measure cervical dilation to monitor labor progress. Early detection of preterm cervical dilation would allow intervention; reducing associated resources, costs and mortality related to preterm labor and premature births. Moreover, a device that can used by a patient in a home environment, can help reduce false labor visits, thereby reducing associated resources, costs, and stress can be advantageous to expectant mothers and their physicians, as well as the prenatal health care providers and industry.

By accurately measuring cervical dilation, with minimal invasiveness and interference, the subject device and methods can address certain problems previously identified for cervical dilation measurements. Resultant advantages provided by the subject invention, include, but are not limited to, facilitation of monitoring of cervical dilation to track labor progression for detecting and intervening in preterm labor, reducing false labor visits and reducing inductions or Caesarean (C-Section) rates. The ability to detect and intervene in pretermlabor would have a positive impact by reducing waste of hospital resources and costs such as unnecessary use of staff and beds.

### BRIEF SUMMARY OF THE INVENTION

The subject invention concerns a camera-based system and device for viewing and measuring cervical dilation during pregnancy and labor. The camera employed in the system and device of the subject invention can be a mono-view or stereo-view camera and can include computer enhancement features to provide at least one of two-dimensional (2D), three-dimensional (3D) imaging, viewing, and measurement capability. Throughout this application, the term camera may also mean “optical imaging” and may be used interchangeably.

The subject invention also includes a method for imaging and measuring cervical dilation which employs a device as described herein.

The useful applications for the subject invention include:
Cervical measurements during pregnancy and labor
General Vaginal Canal Examination
Cervical depth measurement for preterm analysis, and
Vaginal examination for specific conditions (tumors).

The embodiments detailed herein, along with the accompanying drawings, exemplify, but do not limit, the invention, and are understood to convey the concepts and applications of the invention, as well as modifications and alternatives within the scope and spirit thereof, to persons of ordinary skill in the art.

### BRIEF DESCRIPTION OF THE DRAWINGS

FIG. 1 shows a schematic diagram an embodiment of the subject invention and illustrating the components and configuration and relationship of those components in the invention. The first-shaded area illustrates the components that are enclosed within or formed as an integral part of a housing for a device according to the subject invention, and which is part of the system and method of the invention. The second-shaded area illustrates control components within or formed as an integral part of a housing for a device according to the subject invention, and which is part of the system and method of the invention. A third-shaded area illustrates display components within or formed as an integral part of a housing for a device according to the subject invention, and which is part of the system and method of the invention.

FIG. 2 shows a schematic diagram an embodiment of the subject invention comprising a depth sensor, and illustrating the components and configuration and relationship of those components in the invention. The first-shaded area illustrates the components that are enclosed within or formed as an integral part of a housing for a device according to the subject invention, and which is part of the system and method of the invention. The second-shaded area illustrates control components within or formed as an integral part of a housing for a device according to the subject invention, and which is part of the system and method of the invention. A third-shaded area illustrates display components within or formed as an integral part of a housing for a device according to the subject invention, and which is part of the system and method of the invention.

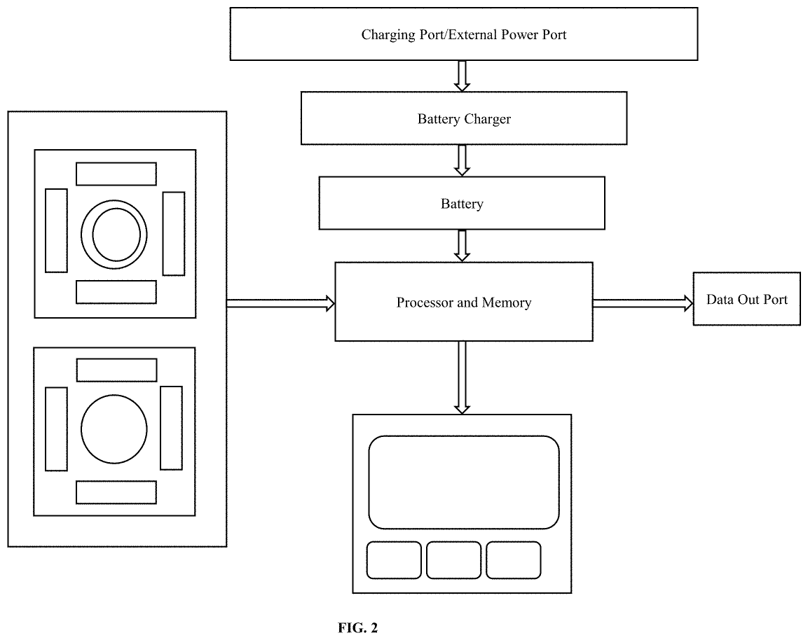

FIG. 3 shows perspective view of a prior art cervical examination probe for monitoring changes in the cervix and screening for cervical cancer.

FIG. 4 shows a SVAKOM rechargeable high-end camera vibrator available in the prior art.

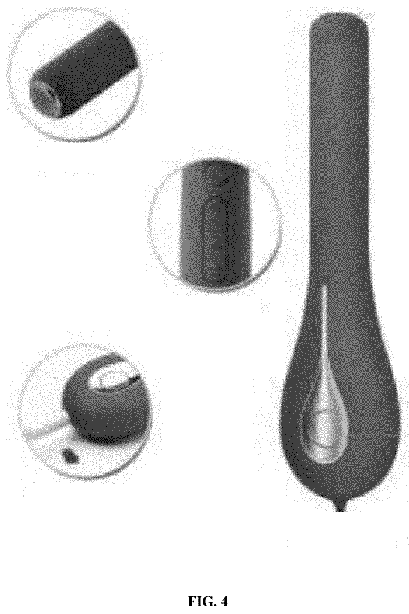

FIG. 5 illustrates a sample embodiment of the subject of the present invention.

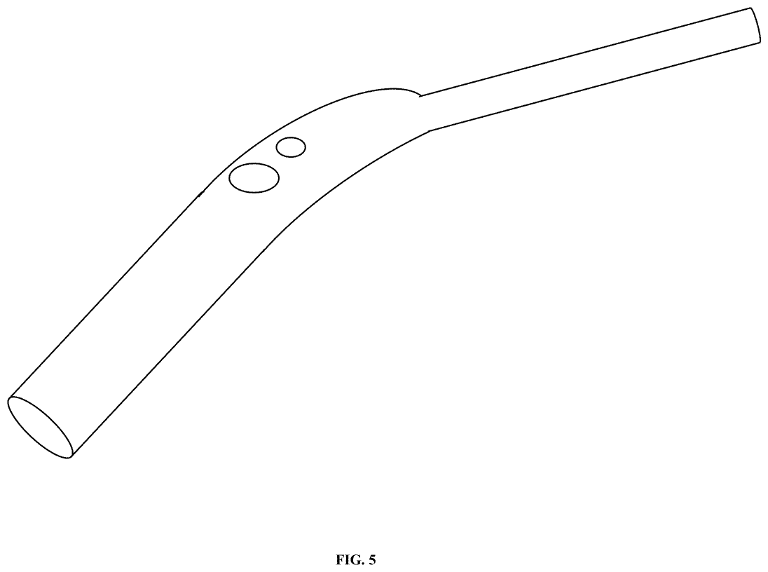

### DETAILED DESCRIPTION OF THE INVENTION

The subject invention comprises a device which employs optical imaging methods to visualize and accurately measure the cervical region of the uterus using two-dimensional (2D) imaging for at least one of 2D, three-dimensional (3D) mapping and 3D imaging. The subject invention also includes the device and peripheral components such as remote display, wired or wireless display, charging or storage components, provided as a system which can be used for imaging the cervix and cervical opening for accurately measuring cervical dilation for assessment of stages of labor in a pregnant woman.

The device of the subject invention advantageously is capable of measuring cervical dilation in the physiologically normal range of about 1-10 cm, with accuracy and variability within the range of 0.1 cm to 0.5 cm, without requiring the device to physically contact the cervix or uterus. The device can be useful to capture images and measurements of the cervical region and cervical dilation for monitoring labor progression in pregnancy. In other embodiments, the device can be useful to capture images and measurements of the cervical region and cervical dilation.

The device of the invention can also be useful for imaging and measuring cervical wall thickness during early stages of pregnancy and can be useful for general vaginal examination.

In one embodiment, a device of the invention can comprise a hand-held wand having a sleek, elongate shape for easy insertion into the vagina and forming a housing for containing the operational components of the device. Preferably the device is devoid of sharp edges or corners to optimize comfort for the patient during use. The wand can include integral therewith, controls for imaging, illumination, and other features, such as focus controls, image capture switches or buttons to “take” an image or picture.

The wand can include an expandable or splayed inserter portion at one end for opening the vaginal canal without the use of a speculum, as well as expanding the surrounding tissue and vagina wall to assist in imaging and to indicate depth of insertion of the device into the vagina.

The housing of the device is preferably made from biocompatible, sterilizable and reusable materials, and may include an outer sterile cover which is removable and replaceable for multiple uses of the device. The sterile cover can be transparent to minimize interference with imaging.

A display interface may be on the device or attached to the device via wired or wireless connection. Controls for imaging and other operational features for using the device for imaging the cervix can be integral with the hand-held wand portion of the device.

The device comprises at least one imaging sensor having a lens and filter optics. The lenses can include focusing or zooming features as desired. Preferably, the device comprises at least one imaging sensor for stereo imaging to generate 3D depth maps and/or 2D images.

Imaging sensor(s) can provide black-and-white and/or color imaging, and/or can provide infrared wavelength imaging.

The device can further include an illumination system for visualizing the area to be imaged and measured.

In another embodiment, the device can be configured having one imaging sensor for 2D imaging with corresponding visual wavelength illuminator and a second imaging sensor configured specifically for depth sensing using specific wavelengths in visual or infrared wavelengths. This configuration shines an array of light, in a dotted or lined pattern that can be processed to generate 3D depth measurements.

The device is capable of generating 3D depth images to measure the cervical opening with or without 2D image overlays.

The measurements from the image generated by the device can be manually or can be automatically processed using an integrated computer or other electronic calculation system which receives the data from the imaging device in real time or input from an electronic storage means.

The 3D depth map can provide additional privacy relative to the 2D imaging.

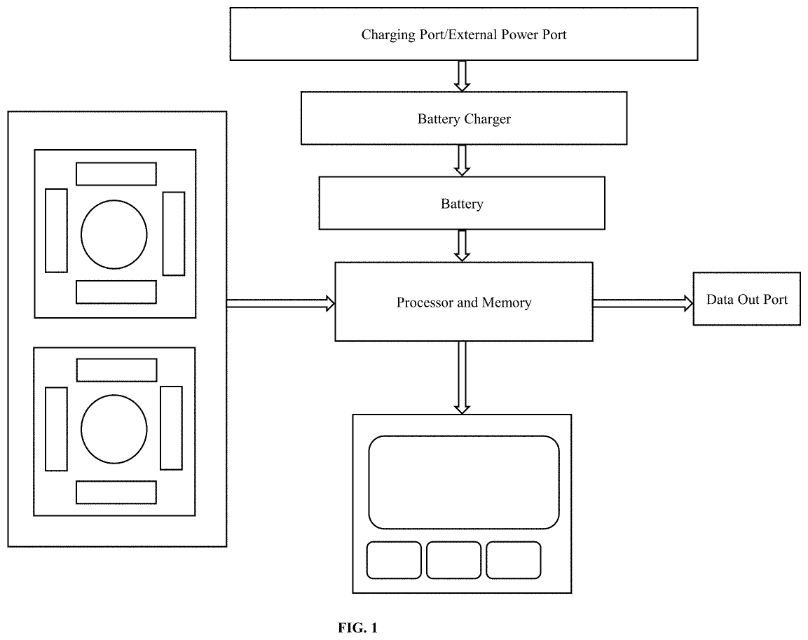

A preferred embodiment of a device of the subject invention is schematically illustrated in FIG. 1, and comprises at least one imaging sensor for imaging the body part of interest.

Illuminating components, or illuminators, are included to provide lighting required for operation of the imaging sensor. These illuminating components can be any lighting source, but are preferably LED illuminators. LED illuminators useful for the subject invention are readily available in the market. Preferably, the LED illuminators provide white light, or can provide light of specific wavelengths, such as light within the infrared wavelength range. More preferably the LED illuminators can generate light of varying wavelengths, including white light or infrared light, and can be adjusted by the user as needed for the desired image being produced.

As shown in FIG. 1, a device of the subject invention is preferably configured such that the illuminating components are provided spatially proximate to the imaging sensor or sensors to optimize the lighting of the visual field and facilitate the imaging procedure.

The image captured by the image sensors, under illumination by the illuminators, is electronically transmitted to an image capture component, e.g., a computer processor and storage device. The image capture components are well known in the art and are readily available on the market. The image capture component is capable of transmitting the image to a display which is provided integral with the device or system of the invention, or a separate component, such as a computer monitor, laptop or smartphone screen, or the like. The display can be integral with (wired) to the image capture component or can be remotely (wirelessly) in communication with the image capture component.

The device can be powered using electrical energy, preferably generated by one or more battery, and more preferably by one or more rechargeable battery.

In a preferred embodiment the device comprises a hand-held wand comprising an elongate body forming a housing for the device components contained within or integral with the housing. The elongate housing has an imaging end for insertion into the vagina or other body cavity, and a control end forming a handle for the user, and comprising controls for operation of the device and the illumination and imaging components. The imaging sensor and illuminating components can be positionally provided at the imaging end of the hand-held wand, which is preferably configured in a shape that can be comfortably inserted into the vagina and capable of visualizing the cervix, as described.

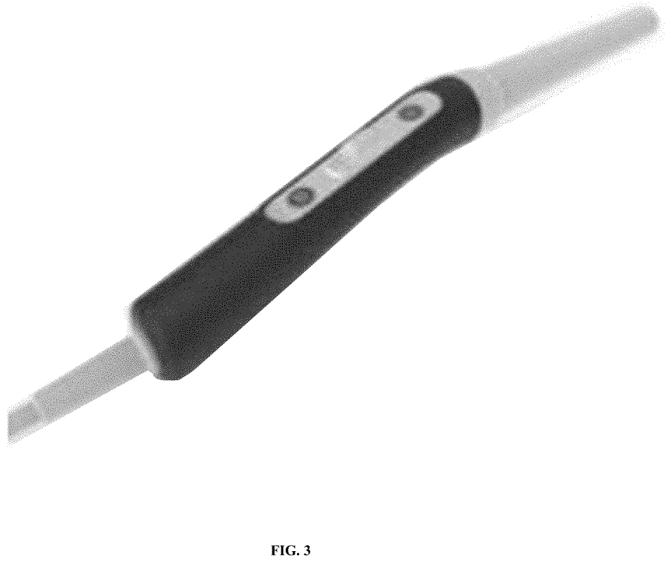

Opposite the imaging end, at the control end of the hand-held wand, the housing can comprise controls for operating the device and for processing of the image captured. For example, the hand-held wand can comprise a switch, buttons, or adjusting dials (including digital adjusting dials) for operating the device. These controls can include, for example, on/off control for the device, imaging sensor or illuminator, imaging operation control (taking a picture or video, focus, or zoom), wavelength selection or adjustment of the illuminating component, or the like. It is understood that the controls can be provided in a user-friendly configuration, for example, as shown in FIG. 3.

The image capture component, which can include an image processor or image storage component or both, can preferably be housed within the body of the hand-held wand. Alternatively, the image capture component can be remote from the wand and in wired or wireless communication with the image sensor or sensors.

The image capture component is, in turn, in communication with a display, such as an image screen forming part of the device or system of the invention. The display can be provided integral with the hand-held wand, but is preferably remote from the wand for purposes of providing a larger viewing screen for the benefit of the user. A display as part of the device or system is optional, as the image can be displayed on a computer or smart-phone screen configured for receiving data or information from the image capture component and displaying an image generated therefrom.

It would be understood that a device or system of the invention can include at least one of (i) a display as part of the device or system and (ii) also include the capability to transmit an image to a computer or smart-phone screen.

In use, a person such as a healthcare worker or other person using the device (including self-examination by the patient) can utilize the device to image the cervix and cervical opening to accurately measure cervical dilation. The wand is inserted into the vagina at a distance such that the cervix can be visualized, the image or images captured by the image capture component, and the information (in readable form as an image) transmitted to a display for real-time assessment and diagnosis.

Advantageously, a device and system according to the subject invention can optically image the cervical opening without contacting the cervix or cervical wall or opening. The display can be part of the device or system or can be transmitted to a computer or smartphone screen for remote viewing and assessment. Alternatively, the image data can be transmitted to a data storage means for remote access and assessment at a later time.

Advantageously, the device and system of the subject invention can be less expensive, use materials readily available, such as electronics and switches for the operational components of the device, and polymeric or silicone-based materials for the housing. The device can be sterilizable and reusable, providing a rugged or robust but simple industrial design, having an optional tethered display or capable of being in wireless communication with remote display components.

The above description and accompanying drawings are provided to describe the invention in a way which enables a person to understand the invention and to make and use the invention. The description is not limiting to the embodiments described and will inform others as to the full scope of the invention to which the inventor is entitled. The drawings are for illustrative purposed only and do not limit the invention. Obvious modifications and alternative embodiments of the invention are included within the full scope and spirit of the invention disclosed in this application.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A device for measuring cervical dilation without contacting the cervix, said device comprising:
a housing forming an elongate hand-held wand having an imaging end and a control end, said imaging end comprising at least one sensor, independently selected from at least one of an image sensor and a depth sensor, and at least one illuminating component; said control end comprising controls for operating said image sensor and illuminating component, and
a power source for operating the image sensors and illuminating component
an image capture component in electronic communication with the image sensors for receiving and processing image data from the image sensor
a display in communication with the image capture component for visualizing the image received from the image sensors.

2. The device of claim 1 wherein at least one sensor is used for a 3d image, but if two sensors are used, they are different, wherein a first sensor is an image sensor and a second sensor is a depth sensor whereby the image sensor and depth sensor can generate a 3d image.

3. The device of claim 1 wherein the two sensors are image sensors to provide a stereo image.

4. A system for measuring cervical dilation, said system comprising:
a device of claim 1, and
at least one peripheral component in communication with the image capture component selected from a computer monitor and a laptop or smartphone screen.

5. A method for measuring cervical dilation, said method comprising:
providing a device of claim 1, and
Imaging the cervix of a pregnant woman to measure and assess dilation.
